(19) United States
(12) Patent Application Publication
(10) Pub. No.: US 2026/0315045 A1
(43) Pub. Date: Oct. 15, 2026

(54) CAPTURE SLEEVES FOR CYTOLOGY BRUSHES

(71) Applicant: Boston Scientific Scimed, Inc., Maple Grove, MN (US)

(72) Inventors: Matthew David SZKOLNICKI, Marlborough, MA (US); Adam McGRAW, Mansfield, MA (US); Andrew James SCHAUBHUT, Bolton, MA (US)

(73) Assignee: Boston Scientific Scimed, Inc., Maple Grove, MN (US)

(21) Appl. No.: 19/643,985

(22) Filed: Apr. 10, 2026

Related U.S. Application Data
(60) Provisional application No. 63/788,886, filed on Apr. 15, 2025.

(51) Int. Cl. A61B 10/02

(52) U.S. Cl. CPC A61B 10/02; A61B 2010/0216

(57) ABSTRACT

A medical device includes a shaft defining a lumen extending therein along a central longitudinal axis of the medical device. The medical device includes a distal tip at a distal end of the shaft and defining a channel within the distal tip and extending substantially along the central longitudinal axis. The distal tip includes a first portion defining a proximal portion of the channel and a second portion defining a distal portion of the channel. The medical device includes a wire movably disposed at least partially in the shaft and the distal tip. The medical device includes an assembly movably coupled to the wire. The assembly is movable through the channel of the distal tip between at least a collapsed configuration and an expanded configuration. The assembly is configured to radially compress into the collapsed configuration when positioned within the distal portion of the channel.

### CROSS-REFERENCE TO RELATED APPLICATION(S)

This application claims the benefit of priority of U.S. Provisional Patent Application No. 63/788,886, filed on Apr. 15, 2025, the entirety of which is incorporated herein by reference.

### FIELD OF INVENTION

This disclosure relates to medical devices for collecting tissue samples, and more particularly to cytology brushes with capture mechanisms to improve sample retention during retraction of the brush.

### BACKGROUND

Various medical devices are often used to collect tissue samples for diagnostic purposes, each of which may be designed to address specific tissue sample collection needs across various medical procedures. For example, cytology brushes are commonly used to collect cells from specific areas within the body for examination. Although these brushes are useful in many applications, they face certain challenges, including potential loss of collected sample material during retraction or otherwise limiting or reducing a size of the sample. There is an increasing demand for more efficient and reliable medical devices, systems, and related methods for tissue sample collection. Various aspects of this application may help to improve sample retention, increase collection capacity, or otherwise help in the sampling of various treatment sites of a subject.

### SUMMARY

Each of the aspects disclosed herein may include one or more of the features of the features described in connection with any of the other disclosed aspects.

In some aspects, the techniques described herein relate to a medical device including a shaft defining a lumen extending therein along a central longitudinal axis of the medical device, a distal tip at a distal end of the shaft and defining a channel within the distal tip and extending substantially along the central longitudinal axis, a wire, and an assembly movably coupled to the wire. The distal tip may include a first portion defining a proximal portion of the channel and a second portion defining a distal portion of the channel. The wire may be movably disposed at least partially in the shaft and the distal tip. The assembly may be movable through the channel of the distal tip between at least a collapsed configuration and an expanded configuration. The assembly may be configured to radially compress into the collapsed configuration when positioned within the distal portion of the channel.

The medical device may include one or more of the following features. The second portion of the distal tip may include a slow-recovery polymer material that is movable between at least a relaxed state and a deformed state. The distal portion of the channel may include a distal opening defined through the second portion of the distal tip, and the distal opening may include a first diameter when in the relaxed state and a second diameter greater than the first diameter when in the deformed state. When in the expanded configuration and positioned at least partially within the proximal portion of the channel, the assembly may be configured to radially expand the distal opening from the relaxed state towards the deformed state. The slow-recovery polymer material may be configured to cause the second portion to return from the deformed state into the relaxed state after a recovery time.

The assembly may be a strut assembly including a plurality of struts and a spring. The spring may be coupled to the plurality of struts, and the spring may be configured to urge the plurality of struts into the expanded configuration when at least a portion of the plurality of struts is positioned distal to the first portion of the channel. The strut assembly may include a joint that is coupled to proximal ends of each of the plurality of struts. As the strut assembly moves toward the expanded configuration, distal ends of each of the plurality of struts may radially expand in at least two directions relative to the joint. The spring may be coupled to an intermediate portion of each of the plurality of struts. The second portion may include a distal surface having a distal opening formed therethrough into the distal portion of the channel defined therein. The distal surface may be formed of a slow-recovery polymer material that is radially expandable between a relaxed state and a deformed state. The strut assembly may be configured to cause the second portion to move at least between the relaxed state and the deformed state in response to proximal retraction of the strut assembly into contact with the distal surface of the second portion and into the distal portion of the channel therein. Proximal retraction of the strut assembly into contact with the first portion of the distal tip may cause the strut assembly to move into the collapsed configuration and into the proximal portion of the channel.

The assembly may be a spring assembly. The spring assembly may include a spring and a catch. The spring may be movably disposed in the channel of the distal tip, and the catch may be secured to a proximal portion of the spring. The wire may be movably coupled to the spring assembly. Movement of the wire may be configured to cause movement of the spring assembly through and relative to the distal tip.

The medical device may further include a brush. The brush may be coupled to the wire and disposed in the channel defined by the distal tip. The brush may include a plurality of bristles that extend radially outward from the wire. The brush may be configured to collect one or more tissue samples when positioned distal of the second portion of the distal tip. The brush may be configured to proximally retract into the distal tip such that the second portion radially encloses the one or more tissue samples

In other aspects, the techniques described herein relate to a medical device including a shaft defining a lumen extending therein along a central longitudinal axis of the medical device, a brush disposed within the shaft and configured to collect tissue particles, a sleeve secured at a distal end of the shaft and disposed radially around the brush, and an actuator movably coupled to the sleeve. The sleeve may be movable at least between a collapsed configuration and an expanded configuration. The sleeve may define a volume when in the expanded configuration that is configured to direct tissue particles toward the brush in a radially inward direction relative to the central longitudinal axis.

The medical device may include one or more of the following features. The actuator may include a syringe in fluid communication with a reservoir at the distal end of the shaft. The medical device may further include a linear actuator disposed in a cavity at the distal end of the shaft, an actuation wire having a distal end coupled to the linear actuator, and a handle including a handle body and the actuator. The actuator may include a spool coupled to a proximal end of the actuation wire, and the spool may be movable relative to the handle body.

In yet further aspects, the techniques described herein relate to a medical device including a handle, a shaft extending distally from the handle along a central longitudinal axis, a distal tip coupled to a distal end of the shaft, a brush disposed within the distal tip and configured to collect tissue particles, and a sleeve disposed within the distal tip and configured to move between at least a retracted position and an extended position. The distal tip may include a first portion and a second portion that is movable relative to the first portion. The handle may include an actuator operably coupled to the sleeve and configured to move the sleeve at least between the retracted position and the extended position.

The medical device may include one or more of the following features. The sleeve may include one or more surface features configured to engage tissue particles. The one or more surface features may include at least one of a protrusion, a ridge, or an adhesive material.

The foregoing general description of the illustrative embodiments and the following detailed description thereof are merely exemplary aspects of the teachings of this disclosure and are not restrictive.

### BRIEF DESCRIPTION OF FIGURES

The accompanying drawings, which are incorporated in and constitute a part of this specification, illustrate exemplary aspects of the disclosure and together with the description, serve to explain the principles of the disclosure.

FIG. 1 depicts a medical device, according to aspects of the disclosure.

FIG. 2 depicts another medical device, according to aspects of the disclosure.

FIGS. 3A, 3B, and 3C each depict a distal portion of respective medical devices, according to aspects of the disclosure.

FIGS. 4 A, 4 B, 4 C, 4 D, 4 E , and FIG. 4 F depict various views of distal portions of another medical device in various stages of operation, according to aspects of the disclosure.

FIGS. 5 A, 5 B, 5 C , and FIG. 5 D depict various views of distal portions of yet another medical device in various stages of operation, according to aspects of the disclosure.

### DETAILED DESCRIPTION

The following description sets forth exemplary aspects of this disclosure. It should be recognized, however, that such description is not intended as a limitation on the scope of this disclosure. Rather, the description also encompasses combinations and modifications to those exemplary aspects described herein.

The terms "proximal" and "distal" are used herein to refer to the relative positions of the components of exemplary medical devices. As used herein, "proximal" refers to a position relatively closer to the exterior of the body or closer to an operator using the medical device. In contrast, "distal" refers to a position relatively further away from the operator using the medical device, or closer to the interior of the body. Several drawings include arrows labeled “P” and “D,” indicating proximal and distal directions, respectively.

As used herein, the terms "comprises," "comprising," "including," "includes," "having," "has," or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term "exemplary" is used in the sense of "example," rather than "ideal."

Further, relative terms such as, for example, "about," "substantially," "approximately," etc., are used to indicate a possible variation of ±10% in a stated numeric value or range. As used herein, the phrase "based on" is understood to be equivalent to the phrase "based at least on," unless indicated otherwise. The term "or" is used disjunctively, such that "at least one of A or B " includes, (A), (B), (A and B), (A or B), (B or A), (A and A), (B and B), etc. It will also be understood that, although the terms first, second, third, etc. are, in some instances, used herein to describe various elements, these elements should not be limited by these terms. These terms are only used to distinguish one element from another.

The medical devices herein may be inserted into a variety of body lumens and/or cavities, such as, for example, the urinary tract or gastrointestinal tract. One or more devices, for example, a cytology brush, may be inserted to collect tissue samples by scraping or brushing the brush against one or more surfaces therein. As the brush is moved, cells or other small tissue fragments adhere to the bristles, allowing for sample collection. It will be appreciated that, unless otherwise specified, bronchoscopes, duodenoscopes, endoscopes, gastroscopes, endoscopic ultrasonography ("EUS") scopes, colonoscopes, ureteroscopes, bronchoscopes, or another delivery device may be used in connection with the features described herein. These delivery devices may be useful, for example, to guide the brush to the target area and review tissue samples therefrom.

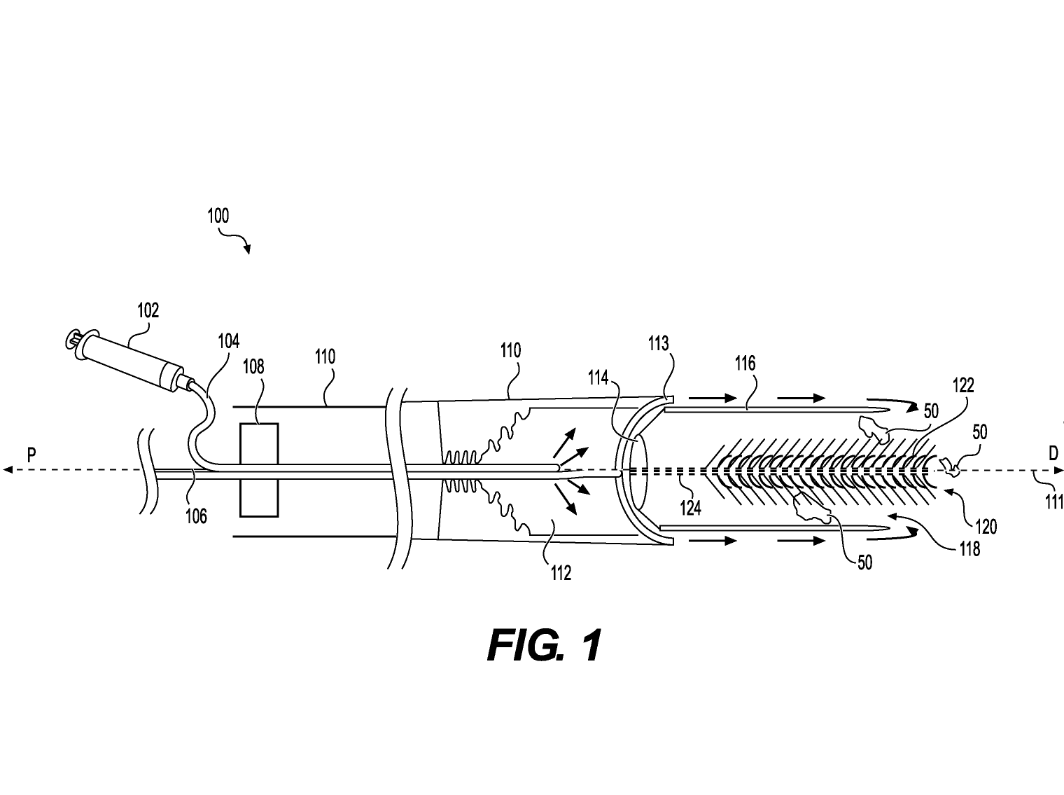

FIG. 1 illustrates a schematic view of a medical device 100, according to some aspects, with a distal portion of the medical device 100 shown as a partial cross-sectional view. The medical device 100 includes an actuator 102 at a proximal end, a shaft 110, a sleeve 116, and a brush 120. As discussed in detail below, the brush 120 is movable relative to one or more of the shaft 110 and the sleeve 116, for example, to obtain one or more tissue samples 50. Furthermore, the sleeve 116 may be movable relative to the shaft 110 and the brush 120, for example, based on manipulation of the actuator 102. For example, the sleeve 116 includes a radially outer surface 116A and a radially inner surface 116B, with the outer and inner surfaces 116A, 116B being moveable relative to one another. The sleeve 116 may roll, for example, with the radially outer surface 116A advancing distally relative to the radially inner surface 116B, for example, as shown by the arrows in FIG. 1. In these aspects, the sleeve 116 may help to protect, enclose, encase, or otherwise at least partially surround one or more portions of the brush 120, for example, to help retain one or more tissue samples 50 on the brush 120.

As mentioned, medical device 100 may include actuator 102 positioned at a proximal end of the medical device 100 relative to a central longitudinal axis 111 thereof. In the example shown, the actuator 102 is a syringe having a plunger 103 slidably disposed therein, the syringe in fluid communication with a tube 104 of the medical device 100. The actuator 102 may be configured to transmit fluid (e.g., air and/or water) contained therein, for example distally through the tube 104 for controlling fluid-driven operations of the medical device 100.

The actuator 102 may provide control over fluid delivery. For example, a user (e.g., medical professional) may actuate the plunger 103 of the syringe (e.g., plunge, deploy, etc.) to generate controlled pressure within the medical device 100. For instance, the user may transmit fluid distally to activate or control distal components of the medical device 100 during a medical procedure, such as expandable elements or tissue collection features. In these aspects, manipulation of the actuator 102 may help to control the position of the sleeve 116, for example, relative to the brush 120.

The medical device 100 may also include an adapter 108 configured to receive the tube 104 therethrough, for example, to the distal end of the medical device 100. The adapter 108 may also be configured to receive one or more wires therethrough, such as a wire 106 of the medical device 100. For example, the wire 106 may extend parallel or otherwise adjacent to the tube 104 through the adapter 108, providing a separate pathway for mechanical actuation within the medical device 100. This arrangement may help to allow for independent control of fluid delivery through the tube 104 and mechanical functions via the wire 106. The adapter 108 may provide a transition point between proximal and distal portions of the medical device 100, serving as a junction where components, such as the tube 104 and the wire 106 can be securely integrated into the shaft 110. This transition point may also incorporate sealing elements to help prevent fluid leakage and maintain the integrity of the device's internal channels. The adapter 108 may be designed to accommodate the different diameters or materials of the proximal components (e.g., the actuator 102) and the distal components (e.g., the shaft 110), helping to provide a smooth and secure connection between these sections of the medical device 100.

The shaft 110 may extend distally from the adapter 108 along the central longitudinal axis 111, and the shaft 110 may help to provide a structural framework for the medical device 100 or otherwise serve as a conduit for various internal components. In some cases, the shaft 110 may be formed of one or more biocompatible materials, such as stainless steel, nitinol, medical-grade polymers, etc., which may be selected for durability and flexibility to navigate through anatomical structures during medical procedures.

The shaft 110 may define or otherwise at least partially enclose a reservoir 112 therein, and the reservoir 112 may be in fluid communication with the tube 104. As discussed in detail below, the reservoir 112 is also in fluid communication with the sleeve 116. In some implementations, the reservoir 112 may provide a variable volume chamber that responds to fluid pressure changes from the actuator 102. Fluid communication between the tube 104 and the reservoir 112 may be customized to help control the distal end of the medical device 100. As fluid pressure is applied or released via the actuator 102, the variable volume chamber of the reservoir 112 may expand or compress accordingly. This expandable or compressible characteristic of the reservoir 112 may be achieved through the use of elastic materials or flexible membranes that deform under pressure yet maintain structural integrity.

The ability of the reservoir 112 to change volume in response to fluid pressure may provide several advantages. In some cases, the reservoir may help to allow for controlled expansion of distal components, such as the sleeve 116, which may be useful for tissue collection and retention. Additionally, the variable volume may help absorb and distribute pressure changes, potentially reducing the risk of damage to collected tissue samples 50 or surrounding anatomical structures during the medical procedure. The reservoir 112 may also serve to modulate fluid pressure throughout the medical device 100, which may enhance the overall performance and control of the device during various medical procedures.

The medical device 100 may include a ferrule 114 positioned near the distal end of the shaft 110. The ferrule 114 may serve as a transitional element between the shaft 110 and the sleeve 116. In some cases, the ferrule 114 may provide a secure connection point between these components. This connection may help ensure structural integrity and proper alignment of the distal components of the medical device 100. The positioning and function of the ferrule 114 may help maintain the overall functionality of the device during medical procedures.

In some aspects, sleeve 116 may extend distally from the ferrule 114. In some implementations, the sleeve 116 may form the radial outermost boundary of a distal portion of the medical device 100, for example, defining a void or capture volume 118 therein. This capture volume 118 may be a specialized space designed to accommodate collected tissue samples 50 and the brush 120. The configuration of the sleeve 116 and its associated capture volume 118 may facilitate the collection or retention of tissue samples during use of the medical device 100.

As mentioned, medical device 100 includes the brush 120, and the brush 120 is movable through, and relative to, at least one of the shaft 110 and the sleeve 116. In the examples shown in the Figures, the brush 120 comprises multiple bristles 122 extending radially outward from a core wire 124. In some cases, one or more bristles 122 may have a length that is larger than a radius of the distal surface 113, such that the brush 120 may substantially fill the capture volume 118 defined by the sleeve 116, and one or more bristles 122 may extend radially beyond the distal surface 113 (e.g., when not constrained by the sleeve 116). This may be useful, for example, to facilitate collection and retention of tissue samples 50 at the distal end of the medical device 100. Furthermore, movement (e.g., distal movement) of the radially outer surface 116A of the sleeve 116 may help to enclose the brush 120, including any tissue samples 50 attached to the brush 120.

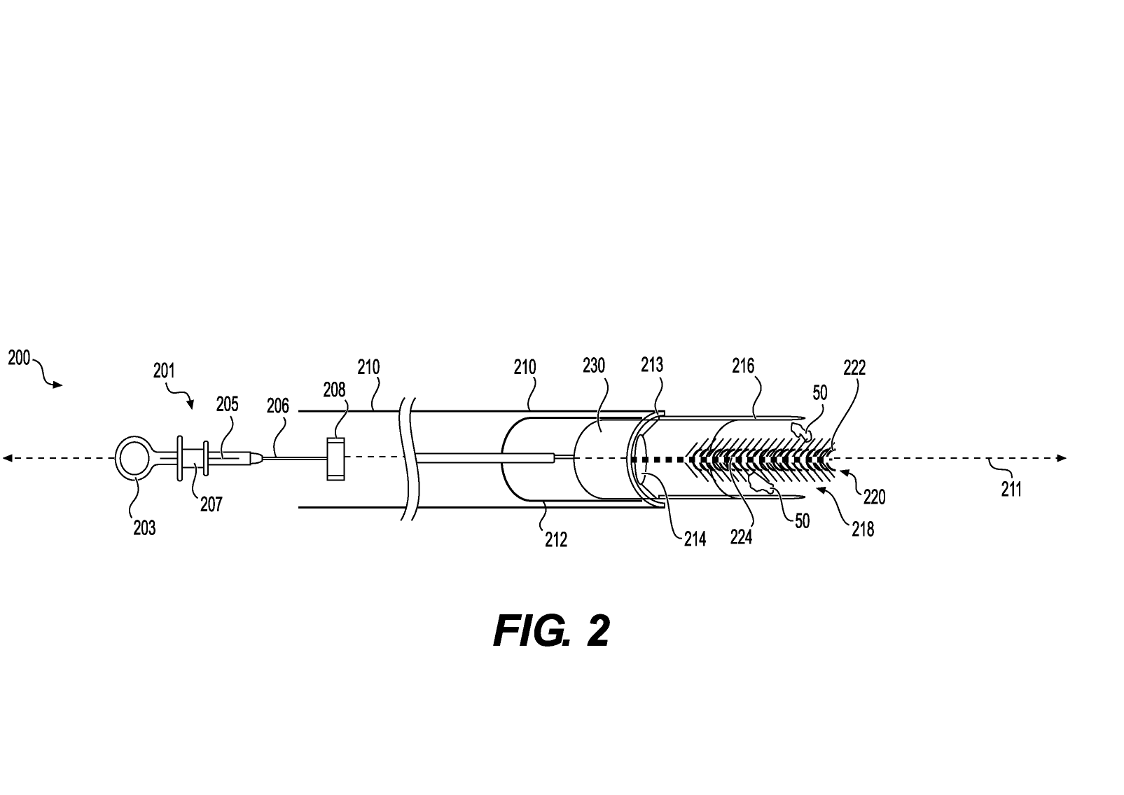

FIG. 2 illustrates a medical device 200 extending along a central longitudinal axis 211, including a partial cross-sectional view of a distal portion of the medical device 200, according to other aspects of this disclosure. The medical device 200 may include a handle 201 positioned at a proximal end. The handle 201 includes a body 203, which may have a slot 205 formed therein. An actuator 207 may be slidably disposed within the slot 205. In some implementations, the actuator 207 may be a spool configured to slide through or along the slot 205 relative to the body 203 between at least a first position and a second position. Nevertheless, it should be understood that other mechanical or non-mechanical actuators may also be used.

The medical device 200 may include a shaft 210 extending distally from the handle 201 to a distal surface 213. The shaft 210 may define a cavity 212 therein. An adapter 208 may be positioned between the handle 201 and the shaft 210, serving as a transition point. A first wire 206 may extend through the medical device 200, passing through the adapter 208 and shaft 210. A ferrule 214 may be positioned near the distal surface 213 of the shaft 210. A sleeve 216 may extend distally from the ferrule 214 and define a void or capture volume 218 therein. A brush 220 may be disposed within the capture volume 218, and the brush 220 includes multiple bristles 222 extending radially outward from a core wire 224 thereof (e.g., to collect tissue samples 50 during a medical procedure).

In some aspects, the medical device 200 may include a linear actuator 230 movably disposed within the shaft 210. This linear actuator 230 may be movably coupled (e.g., directly or indirectly) to the actuator 207, for example via the wire 206, creating a mechanical linkage between the handle 201 and the distal components of the medical device 200. The linear actuator 230 may convert longitudinal motion of the actuator 207 within the slot 205 into corresponding movement at the distal end of the medical device 200. As the user manipulates the actuator 207, for example, sliding the actuator 207 proximally or distally through the slot 205, this motion is transmitted through the linear actuator 230. For example, the linear actuator 230 may move proximally or distally within the shaft 210, in response to movement of the actuator 207, which in turn may affect the position of the sleeve 216 relative to the brush 220. For example, distal movement of the linear actuator 230 may move at least a portion of the linear actuator 230 into the sleeve 216, which may cause the radially outer portion of the sleeve 216 to move distally, for example, becoming part of the radially inner portion of the sleeve 216. As discussed above, distal movement of the linear actuator 230 and the corresponding distal movement of the sleeve 216 may help to enclose one or more portions of the brush 220, along with one or more tissue samples 50.

In some aspects, mechanical coupling between the linear actuator 230 and the sleeve 216 may allow for precise control over tissue collection and retention. For example, distal movement of the linear actuator 230 along the central longitudinal axis 211 may cause the sleeve 216 to extend distally over the brush 220, which may encapsulate collected tissue samples 50. Conversely, proximal movement may retract the sleeve 216, exposing more of the brush 220 for tissue samples 50 collection. By manipulating the actuator 207, the user can adjust the relative positions of the sleeve 216 and brush 220 in real-time during the medical procedure.

In some implementations, the sleeve 216 may define a volume containing a fluid, which may allow the sleeve 216 to rotate or roll relative to the linear actuator 230. As the linear actuator 230 moves proximally or distally, contact with an inner surface of the sleeve 216 may cause rotation or rolling of the sleeve 216 relative to the linear actuator 230. This may be useful, for example, to guide tissue samples 50 in a radially inward direction relative to the central longitudinal axis 211 and toward the brush 220 for collection during medical procedures. The brush 220 may be movable relative to the sleeve 216, enabling extension from and retraction into the capture volume 218 for tissue collection. Medical device 200 therefore may be configured to collect and retain tissue samples 50, for example, through movement of the brush 220 and the sleeve 216, which can be controlled by the actuator 207 and linear actuator 230. Although not shown, the brush 220 may be coupled to one or more actuation wires or control wires, such that the brush 220 may be longitudinally movable and rotatable relative to the shaft 202. For example, medical device 200 may include at least one wire similar to or same as the wire 106 of medical device 100 (FIG. 1).

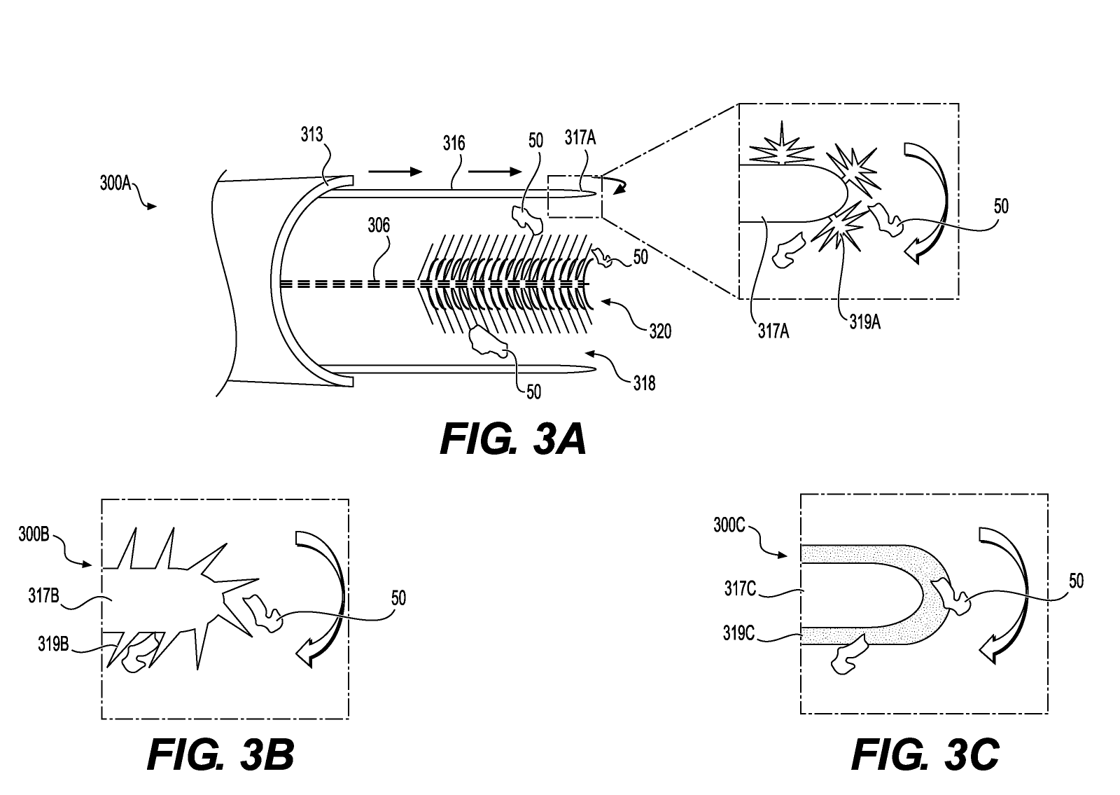

FIGS. 3A-3C illustrate sectional views of three different implementations of medical devices 300A, 300B, and 300C, respectively, each showing alternative sleeve designs with varying surface features. These surface features may be configured to enhance tissue adhesion and capture during medical procedures. In each implementation, the medical device may include a brush 320 disposed within a capture volume 318 defined by a sleeve 316, which may be used to collect one or more tissue samples 50. The various surface features depicted in FIGS. 3A-3C may, for example, be useful to improve the efficiency of tissue sample collection and/or retention during medical procedures.

FIG. 3A illustrates a perspective view of a medical device 300A, including a capture sleeve 316 that comprises a distal portion 317A having one or more surface features 319A configured to engage tissue samples 50. The surface feature 319A may include an arm, barb, protrusion, etc. shaped and sized to direct tissue samples 50 toward a central longitudinal axis. In some implementations, the surface feature 319A may help to guide or otherwise urge tissue samples 50 radially inward toward the brush 320 disposed within the capture volume 318 defined by the sleeve 316. This configuration may help to enhance the collection and retention of tissue samples during medical procedures by facilitating the movement of tissue samples 50 towards the brush 320 for capture.

FIG. 3B illustrates another example of a surface feature 319B on a distal portion 317B of a medical device 300B. The surface features 319B may include one or more ridges, spikes, teeth, etc. disposed on the surface of the distal portion 317B. In some implementations, the surface feature 319B may include a set of teeth that roll inward as the distal portion 317B moves distally, which may facilitate capturing and retaining tissue samples 50 during operation of the medical device 300B.

FIG. 3C illustrates yet another example of a surface feature 319C on a distal portion 317C of a medical device 300C. In this example, the surface feature 319C may include an adhesive (e.g., adhesive hydrogel), a coating with one or more adhesive properties, etc., which may help to increase adhesion with tissue samples 50. The surface feature 319C may include, for example, one or more chemical adhesives, such as methacrylate and/or cyanoacrylate. In some cases, the surface feature 319C may also include lubricating polyethylene glycol (PEG) or polyvinyl alcohol (PVA) hydrogels to help reduce self-adhesion or to help reduce overall friction during the medical procedure.

Any of the surface features 319A, 319B, 319C described herein may be used alone or in combination and may extend along one or more portions of the entire length of the sleeve 316. In some examples, one or more surface features 319A, 319B, 319C may be positioned along one or more portions of the surface of the sleeve 316. In some aspects, one or more surface features 319A, 319B, 319C may be positioned on sleeve 316 between distal surface 313 of the shaft 310 and the distalmost end of the sleeve 316.

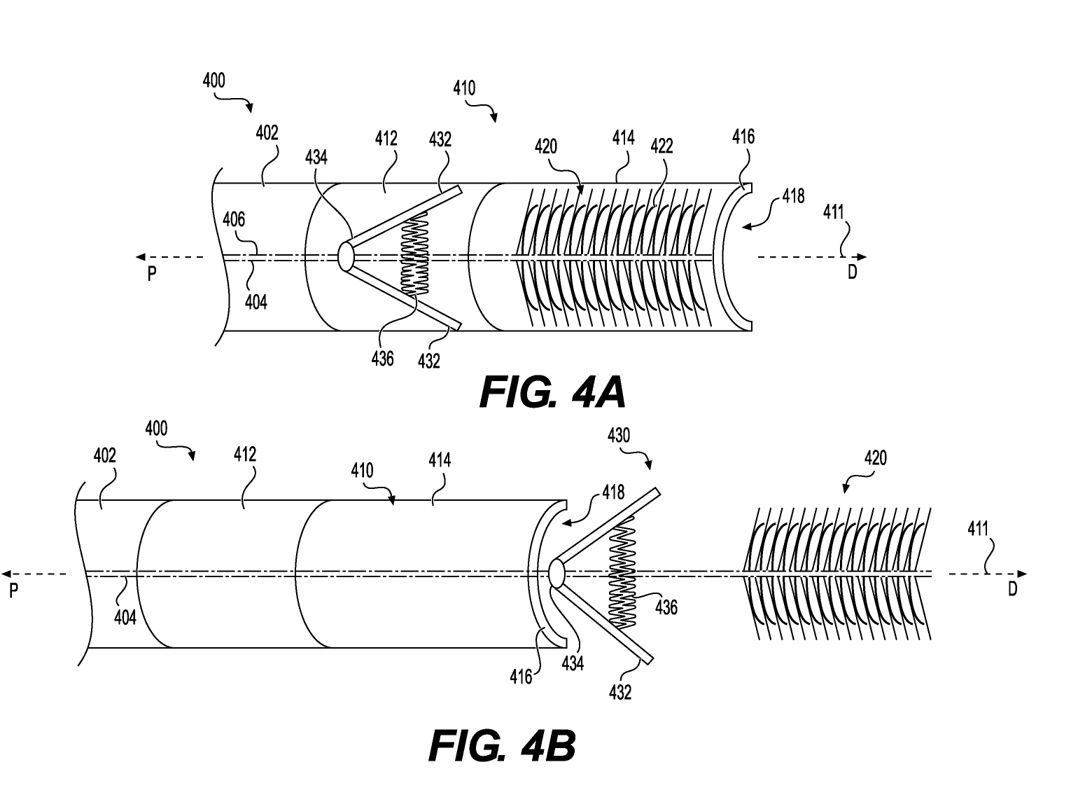

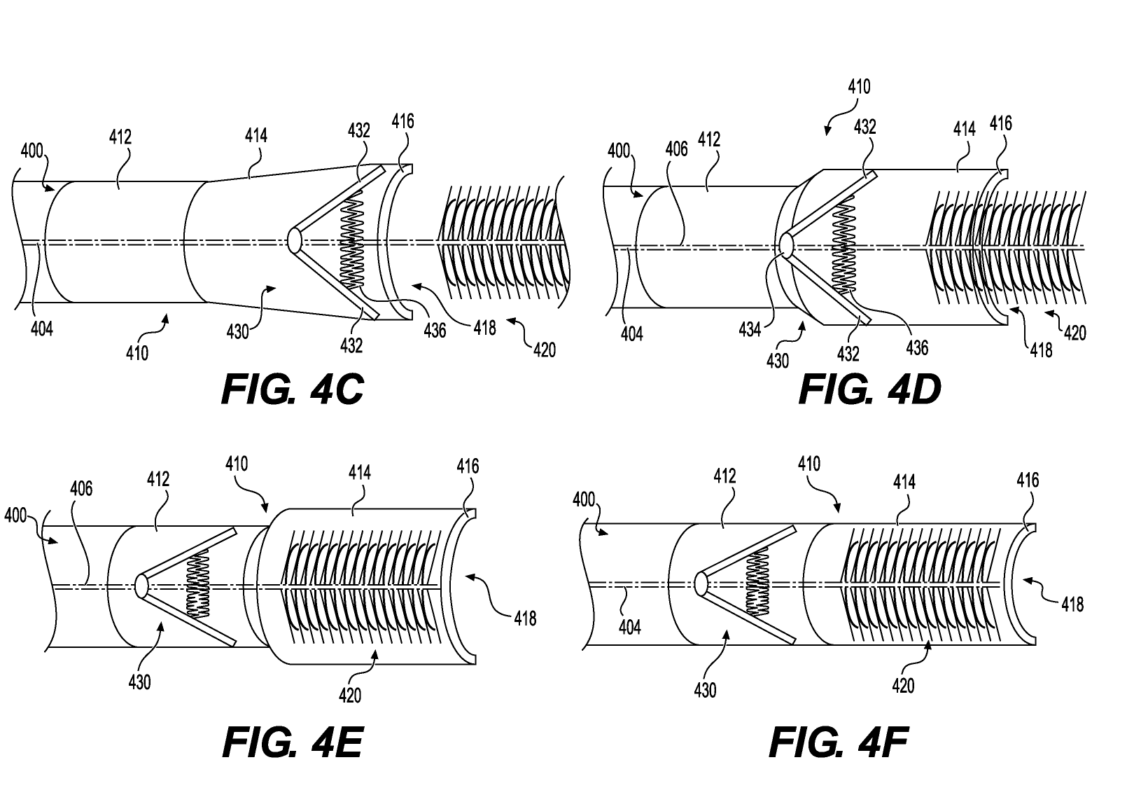

FIGS. 4A-4F illustrate various cross-sectional views of a distal portion of a medical device 400 in various stages of operation, according to aspects of the disclosure.

FIG. 4A shows a sectional view of the medical device 400 in a relaxed state, which may include a shaft 402 defining a lumen therein extending along a central longitudinal axis 411 of the medical device 400. The shaft 402 may extend distally to a distal tip 410 of the medical device 400 positioned at the distal end. The medical device 400 may also include at least one wire 404 that extends through the shaft 402. The wire 404 may be coupled to a brush 420 having a plurality of bristles 422. For example, movement of the wire 404 may help to control movement of the brush 420 and collect a tissue sample at the distal end of the medical device 400.

In some aspects, the distal tip 410 may include a first portion 412 and a second portion 414. For example, the first portion 412 may be positioned proximal of the second portion 414 at the distal end of the medical device 400 relative to the central longitudinal axis 411. The first portion 412 may be formed of one or more rigid materials, such as, for example, stainless steel, nitinol, or rigid polymers like polyetheretherketone (PEEK) or polyimide, which may provide structural stability or support. The second portion 414 may be constructed from one or more flexible materials, such as, for example, silicone, polyurethane, or thermoplastic elastomers (TPEs), which may allow for controlled deformation and expansion. In some cases, the second portion 414 may comprise a slow-recovery polymer material, such as shape memory polyurethane or polylactic acid (PLA), that may maintain an expanded state for a period of time before gradually returning to its original shape. The transition between the first portion 412 and second portion 414 may be achieved through material gradients or composite structures to help ensure a smooth flexibility profile along the distal tip 410.

The distal tip 410 may define a lumen or channel therein, for example extending substantially parallel to the central longitudinal axis 411. The first portion 412 may define a proximal portion of the channel (e.g., rigid portion), and the second portion 414 may define a distal portion of the channel (e.g., flexible portion). As shown, the second portion 414 may have a distal opening 418 extending through a distal surface 416 and into the channel defined in the distal tip 410. In this example, the internal diameter of the channel in the second portion 414 may flex or expand, such that the distal opening 418 is radially expandable or flexible at least between a relaxed state and an expanded state. As discussed in detail below, the distal opening 418 may transition to the expanded state in response to external force.

In some aspects, the channel may extend continuously from the first portion 412 through the second portion 414, for example, to provide a pathway for one or more components, for example, such as the brush 420 or a strut assembly 430 (discussed in detail below) to move through the distal tip 410. The transition between the first portion 412 and second portion 414 may be gradual or abrupt, depending on the desired flexibility profile of the distal tip 410. The second portion 414 may be configured to expand radially outward in response to various stimuli, such as the deployment of the strut assembly 430 or the movement of the brush 420 through the channel defined therethrough.

The distal opening 418 may serve as an entrance and exit for the brush 420 during medical procedures. For example, when the second portion 414 is in the relaxed state, the distal opening 418 may have a first diameter, and when the second portion 414 is in the expanded state, the distal opening 418 may have a second diameter greater than the first diameter. This expansion capability may help to allow for the collection of larger tissue samples and/or facilitate retraction of the brush 420 with samples. In the example shown, the distal opening 418 is substantially circular and, in other examples, the distal opening 418 may have different shapes and contours. The shape or size of the distal opening 418 may be based on material properties of the second portion 414 and/or specific needs to perform any given medical procedure. As such, material properties of the second portion 414 (e.g., elastomer material properties) may be useful to facilitate navigation through complex anatomical structures during medical procedures.

As mentioned above, the medical device 400 may include the strut assembly 430. The strut assembly 430 may be movably disposed within the channel of the distal tip 410. For example, the strut assembly 430 may translate in a proximal direction and/or a distal direction, for example, through and relative to the distal tip 410. The strut assembly 430 may have a plurality of struts 432 configured to radially expand or collapse, for example, between an expanded configuration and a collapsed configuration, respectively. In the collapsed configuration, the strut assembly 430 may be shaped and sized to fit within the channel of the distal tip 410. In some examples, the strut assembly 430 may self-deploy into the expanded configuration, and, in other examples, the strut assembly 430 may have a mechanism for selective deployment. Each of the struts 432 may be formed of a shape memory material, such as nitinol, allowing for self-expansion when unconstrained. The number of struts 432 may vary depending on the desired radial force and expansion characteristics. For example, the strut assembly 430 may include two, three, four, or more struts 432. Each of the struts 432 may have a uniform width along its length, or one or more of the struts 432 may have a varying width, which may help to create different expansion profiles of the strut assembly 430.

The strut assembly 430 may include a joint 434 coupled to a spring 436. The spring 436 may provide a biasing force to assist in deployment of the struts 432 (e.g., radially expand). In some examples, the spring 436 may radially urge the struts 432 into the expanded configuration when at least a portion of the struts 432 is positioned outside of the distal tip 410. The joint 434 may be configured to pivot or otherwise move the plurality of struts 432 between the expanded configuration (e.g., unconstrained or deployed position) into the collapsed configuration (e.g., restrained position). The joint 434 may be a living hinge formed integrally with the struts 432. The spring 436 may be a coil spring, leaf spring, or other suitable biasing element. In other examples, the medical device 400 may include another actuation wire (not shown) that extends through the shaft 402 and is movably coupled to the joint 434, such that moving the wire (e.g., proximally or distally) may correspond to movement of the joint 434. This may be useful to independently control movement for each of the brush 420 and the strut assembly 430.

FIG. 4B shows the medical device 400 in a fully deployed position, with each of the strut assembly 430 and the brush 420 positioned distally of the second portion 414 of the distal tip 410. In this example, the strut assembly 430 moves distally through the distal tip 410 from a collapsed configuration constrained within the channel as shown in FIG. 4A, and the strut assembly 430 radially expands when positioned outside of the distal tip 410 (e.g., moved distally through the distal opening 418). As the strut assembly 430 moves through the distal opening 418, the radial constraint on the struts 432 is released and thereby allows the strut assembly 430 to expand radially outward into the fully deployed position shown in FIG. 4B.

In operation, the strut assembly 430 in the expanded configuration may be proximally retracted through the distal opening 418 into the channel defined by the distal tip 410. As the strut assembly 430 moves proximally from the second portion 414 into the first portion 412, the second portion 414 may gradually return to its original diameter. This delayed recovery may allow time for the brush 420 to retract with collected samples before the second portion 414 fully contracts, which may improve sample retention during retraction or withdrawal of the medical device 400. For example, the second portion 414 may have delayed recovery in moving from the expanded configuration toward the collapsed configuration over a recovery time, which may be based on the specific shape and/or material composition forming the second portion 414. In some cases, the recovery time may be adjustable or customizable for specific medical procedures, for example such that the recovery period of time is in a range from, e.g., about 1 second to about 600 seconds, about 1 second to about 500 seconds, about 1 second to about 250 seconds, about 1 second to about 100 seconds, about 1 second to about 50 seconds, about 1 second to about 25 seconds, about 1 second to about 10 seconds, about 1 second to about 5 seconds, or about 1 second to about 2.5 seconds.

FIG. 4C illustrates this proximal retraction of the strut assembly 430 in the expanded state, which may move the strut assembly 430 into contact with the distal surface 416 and cause the second portion 414 to flex radially outward, increasing the local diameter of the distal tip 410. Additionally, the expansion may correspond to the brush 420 being extended from the distal tip 410, for example, to obtain one or more tissue samples.

FIG. 4D shows continued proximal retraction of the strut assembly 430 and the brush 420 into the channel defined by the distal tip 410. As shown, this may cause the second portion 414 of the distal tip 410 to become fully expanded or flexed, receiving each of the strut assembly 430 and at least part of the brush 420 therein.

FIG. 4E illustrates further proximal retraction of the strut assembly 430 into the first portion 412 of the distal tip 410. This may cause the strut assembly 430 to move from the expanded configuration into the collapsed configuration radially constrained within the first portion 412. This may initialize or start the delayed recovery time, for example the second portion 414 of the distal tip 410 may now slowly return to the original shape and size after removal of the strut assembly 430.

FIG. 4F shows the medical device 400 in the fully retracted state after the recovery time. As shown, the second portion 414 has returned to the original diameter (e.g., relaxed or rest state), which may cause the distal tip 410 to radially secure tissue samples collected on the brush 420.

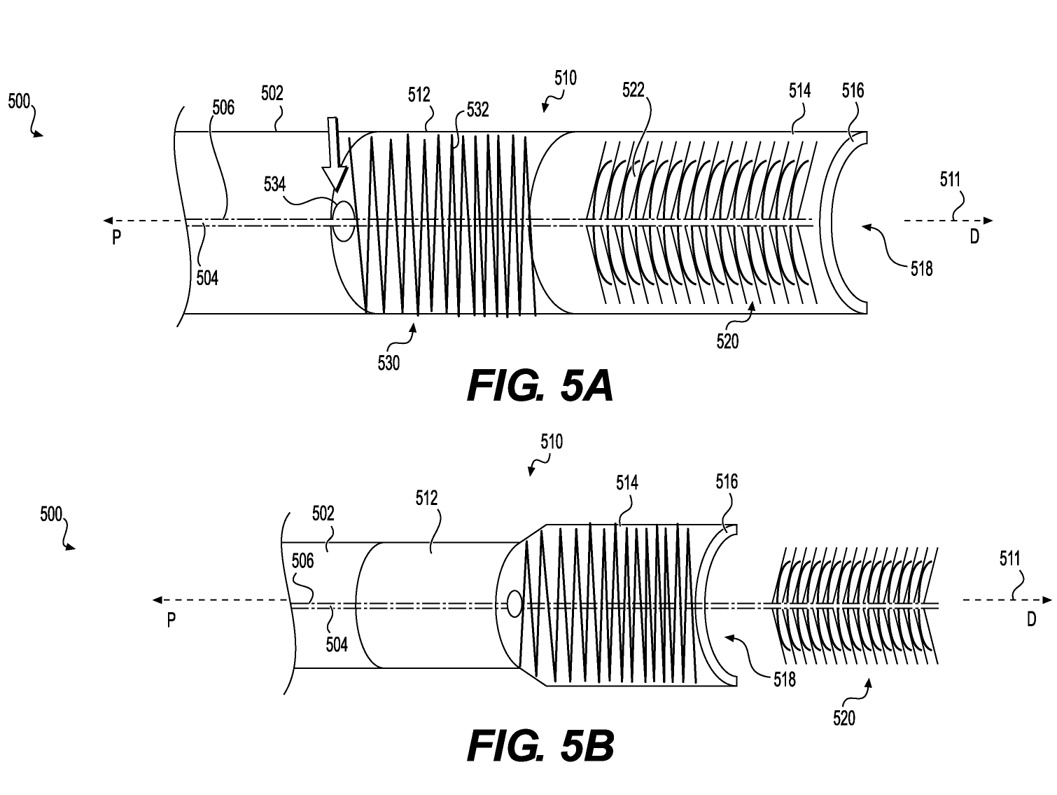

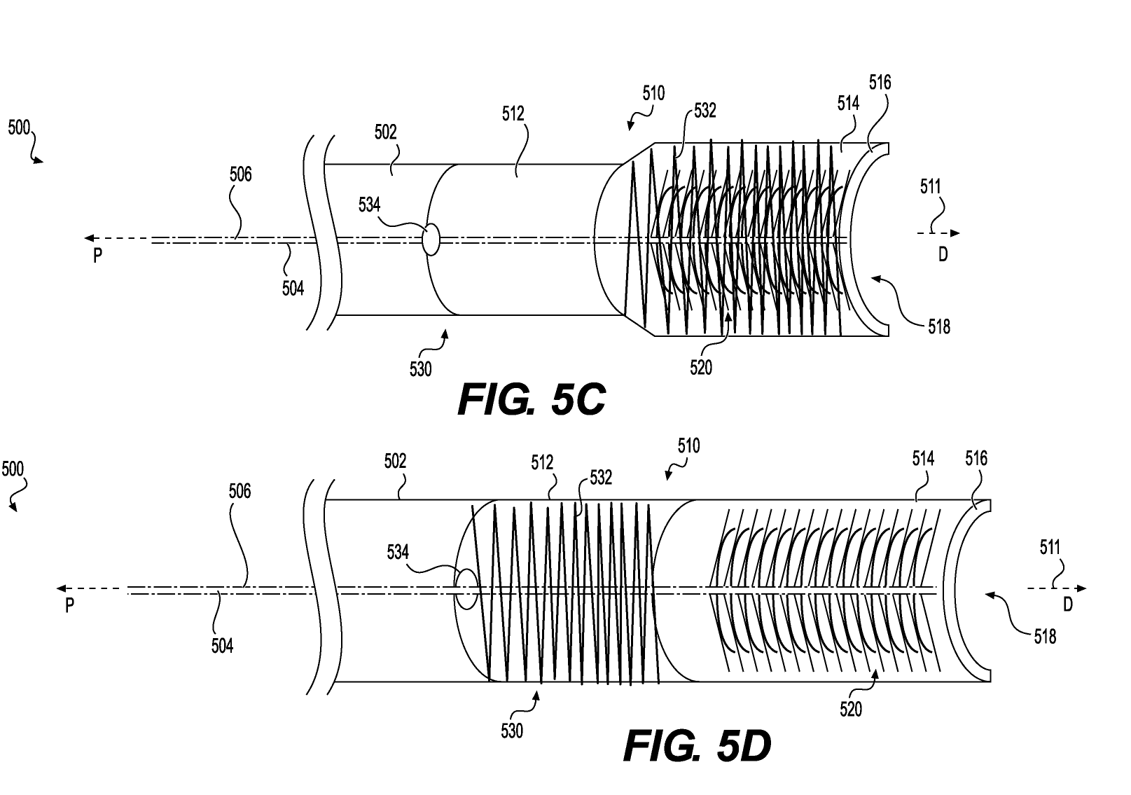

FIGS. 5A-5D depict various views of a medical device 500 according to aspects of the disclosure. The medical device 500 may include a shaft 502 extending distally along a central longitudinal axis 511 of the medical device 500, for example, to a distal tip 510 positioned at a distal end of the medical device 500. The distal tip 510 may include a first portion 512 and a second portion 514. In some examples, the first portion 512 is formed of one or more rigid materials, and the second portion 514 is formed of one or more flexible materials.

FIG. 5A illustrates a sectional view of the medical device 500, showing the arrangement of components along the central longitudinal axis 511. The medical device 500 may include a first wire 504 and a second wire 506 extending through the shaft 502. A spring assembly 530 may be positioned within a channel defined by the distal tip 510, for example at a distal end of the medical device 500. When deployed, the spring assembly 530 may expand radially, in turn causing the second portion 514 of the distal tip 510 to stretch or otherwise expand and increase local diameter of the distal opening 518. As discussed in detail below, the spring assembly 530 includes one or more springs 532 and a catch 534, for example, to couple the one or more springs 532 to one or more wires.

As shown in FIG. 5A, when the spring assembly 530 is positioned within the first portion 512 of the distal tip 510, the spring assembly 530 may be maintained in a collapsed state or configuration. The first portion 512 may provide structural support to constrain the spring 532 in its compressed form. As the spring assembly 530 translates distally through the channel defined within the distal tip 510, it may transition from the first portion 512 into the second portion 514.

As shown in FIG. 5B, when the spring assembly 530 is positioned within the distal portion of the channel defined by the second portion 514, the spring 532 may radially expand into an expanded state or configuration. This expansion may occur due to release of stored potential energy in the compressed spring 532, as well as the reduced structural constraints provided by the second portion 514 compared to the first portion 512. The radial expansion of the spring 532 may cause a corresponding expansion of the second portion 514, which may increase the diameter of the distal opening 518 defined therethrough.

Movement of the spring assembly 530 may be controlled by at least one of the wires 504, 506. In some implementations, one of these wires may be coupled to the catch 534, allowing for precise control over the deployment and retraction of the spring assembly 530. The transition of the spring assembly 530 from its collapsed state to its expanded state may be gradual, for example as the spring assembly 530 moves from the first portion 512 to the second portion 514, or it may be more abrupt, for example depending on the specific geometric profile of the spring assembly 530 and/or material properties of the second portion 514.

In some aspects, the medical device 500 may include a mechanism for controlled retraction of the spring assembly 530. For example, the catch 534 may be operably coupled to the second wire 506, which extends through the shaft 502. The user may actuate the catch 534 via the second wire 506 to initiate the retraction process of the spring assembly 530. Upon actuation, the catch 534 may engage with the spring 532, causing the spring assembly 530 to transition from its expanded configuration to a collapsed configuration. As the spring assembly 530 collapses, it may be proximally retracted into the first portion 512 of the distal tip 510. This retraction may be facilitated by the second wire 506, which may be configured to transmit a pulling force from a proximal end of the medical device 500 to the catch 534.

The first portion 512 may be shaped and sized to accommodate the collapsed spring assembly 530, providing a housing for the assembly 530 when not deployed. The second portion 514 may exhibit a delayed response to the retraction of the spring assembly 530. This delay in recovery may result in the distal opening 518 formed in the distal surface 516 maintaining an increased diameter for a period after the spring assembly 530 has been retracted, for example, to help receive the brush 520 and any tissue samples when the brush 520 is proximally retracted.

FIG. 5B depicts the medical device 500 in a deployed state, with the spring assembly 530 having the radially expanded spring 532 positioned in the second portion 514 of the distal tip 510, in turn causing the second portion 514 to stretch the distal surface 516 and distal opening 518 formed therethrough. A brush 520 is shown in FIG. 5B as positioned distally of the distal tip 510, for example to collect one or more tissue samples.

FIG. 5C shows the medical device 500 in the flexed state according to some aspects. As shown, the distal opening 518 may have increased diameter when in the flexed state as compared to diameter in the relaxed state (FIG. 5A). In some examples, the user may proximally retract the brush 520, including any tissues sample(s), through the distal opening 518 of the distal surface 516 having the increased diameter, and the second portion 514 may have delayed recovery to the relaxed state.

FIG. 5D shows the medical device 500 after the brush 520 has been fully retracted. The properties of the second portion 514 may allow it to maintain its expanded state for a recovery time, for example, before gradually returning to the original diameter defining the relaxed state.

This disclosure provides various embodiments of a medical device for collecting tissue samples. These embodiments may include variations in sleeve materials, surface features, deployment mechanisms, and expandable components. The disclosed medical devices may offer benefits such as improved tissue collection efficiency, enhanced sample retention, and reduced trauma to surrounding tissues during medical procedures. The combination of flexible portions with slow-recovery properties and expandable mechanisms may allow for more precise control over the tissue collection process, potentially increasing the quality and quantity of obtained samples.

Various aspects of the principles and techniques described herein may be used in minimally invasive diagnostic and therapeutic procedures across various medical specialties. The ability to collect multiple tissue samples without device removal, coupled with the potential for larger sample sizes, may contribute to more efficient or effective biopsy procedures. It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

1. A medical device comprising:
a shaft defining a lumen extending therein along a central longitudinal axis of the medical device;
a distal tip at a distal end of the shaft and defining a channel within the distal tip and extending substantially along the central longitudinal axis,
wherein the distal tip includes a first portion defining a proximal portion of the channel and a second portion defining a distal portion of the channel;
a wire movably disposed at least partially in the shaft and the distal tip; and
an assembly movably coupled to the wire, wherein the assembly is movable through the channel of the distal tip between at least a collapsed configuration and an expanded configuration, and
wherein the assembly is configured to radially compress into the collapsed configuration when positioned within the distal portion of the channel.

2. The medical device of claim 1, wherein the second portion of the distal tip comprises a slow-recovery polymer material that is movable between at least a relaxed state and a deformed state.

3. The medical device of claim 2, wherein the distal portion of the channel includes a distal opening defined through the second portion of the distal tip, wherein the distal opening has a first diameter when in the relaxed state and a second diameter greater than the first diameter when in the deformed state.

4. The medical device of claim 3, wherein, when in the expanded configuration and positioned at least partially within the proximal portion of the channel, the assembly is configured to radially expand the distal opening from the relaxed state towards the deformed state.

5. The medical device of claim 3, wherein the slow-recovery polymer material is configured to cause the second portion to return from the deformed state into the relaxed state after a recovery time.

6. The medical device of claim 1, wherein the assembly is a strut assembly comprising a plurality of struts and a spring, wherein the spring is coupled to the plurality of struts, and wherein the spring is configured to urge the plurality of struts into the expanded configuration when at least a portion of the plurality of struts is positioned distal to the first portion of the channel.

7. The medical device of claim 6, wherein the strut assembly comprises a joint that is coupled to proximal ends of each of the plurality of struts, and
wherein, as the strut assembly moves toward the expanded configuration, distal ends of each of the plurality of struts radially expand in at least two directions relative to the joint.

8. The medical device of claim 6, wherein the spring is coupled to an intermediate portion of each of the plurality of struts.

9. The medical device of claim 6, wherein the second portion comprises a distal surface having a distal opening formed therethrough into the distal portion of the channel defined therein, wherein the distal surface is formed of a slow-recovery polymer material that is radially expandable between a relaxed state and a deformed state.

10. The medical device of claim 9, wherein the strut assembly is configured to cause the second portion to move at least between the relaxed state and the deformed state in response to proximal retraction of the strut assembly into contact with the distal surface of the second portion and into the distal portion of the channel therein.

11. The medical device of claim 10, wherein proximal retraction of the strut assembly into contact with the first portion of the distal tip causes the strut assembly to move into the collapsed configuration and into the proximal portion of the channel.

12. The medical device of claim 1, wherein the assembly is a spring assembly, wherein the spring assembly includes a spring and a catch, wherein the spring is movably disposed in the channel of the distal tip, and wherein the catch is secured to a proximal portion of the spring.

13. The medical device of claim 12, wherein the wire is movably coupled to the spring assembly, and wherein movement of the wire is configured to cause movement of the spring assembly through and relative to the distal tip.

14. The medical device of claim 1, further comprising:
a brush coupled to the wire and disposed in the channel defined by the distal tip, wherein the brush includes a plurality of bristles that extend radially outward from the wire.

15. The medical device of claim 14, wherein the brush is configured to collect one or more tissue samples when positioned distal of the second portion of the distal tip, and wherein the brush is configured to proximally retract into the distal tip such that the second portion radially encloses the one or more tissue samples.

16. A medical device comprising:
a shaft defining a lumen extending therein along a central longitudinal axis of the medical device;
a brush disposed within the shaft and configured to collect tissue particles;
a sleeve secured at a distal end of the shaft and disposed radially around the brush, and
an actuator movably coupled to the sleeve, wherein the sleeve is movable at least between a collapsed configuration and an expanded configuration,
wherein the sleeve defines a volume when in the expanded configuration that is configured to direct tissue particles toward the brush in a radially inward direction relative to the central longitudinal axis.

17. The medical device of claim 16, wherein the actuator comprises a syringe in fluid communication with a reservoir at the distal end of the shaft.

18. The medical device of claim 16, further comprising:
a linear actuator disposed in a cavity at the distal end of the shaft;
an actuation wire having a distal end coupled to the linear actuator; and
a handle including a handle body and the actuator, wherein the actuator comprises a spool coupled to a proximal end of the actuation wire, and wherein the spool is movable relative to the handle body.

19. A medical device comprising:
a handle;
a shaft extending distally from the handle along a central longitudinal axis;
a distal tip coupled to a distal end of the shaft, the distal tip comprising a first portion and a second portion that is movable relative to the first portion;
a brush disposed within the distal tip and configured to collect tissue particles; and
a sleeve disposed within the distal tip and configured to move between at least a retracted position and an extended position,
wherein the handle comprises an actuator operably coupled to the sleeve and configured to move the sleeve at least between the retracted position and the extended position.

20. The medical device of claim 19, wherein the sleeve comprises one or more surface features configured to engage tissue particles, the one or more surface features comprising at least one of a protrusion, a ridge, or an adhesive material.
